(19) United States
(12) Patent Application Publication
(10) Pub. No.: US 2024/0252770 A1
(43) Pub. Date: Aug. 1, 2024

(54) INHALER WITH AN ADHERENCE/COMPLIANCE MONITOR

(71) Applicant: VECTURA DELIVERY DEVICES LIMITED, Wiltshire (GB)

(72) Inventors: Philip SMITH, Cambridgeshire (GB); Philip SWANBURY, Cambridgeshire (GB); Andreas MELINIOTIS, Cambridgeshire (GB); Danny MORRIS, Staffordshire (GB); Jack DUNKLEY, Staffordshire (GB); Mark HEYNES, Staffordshire (GB)

(73) Assignee: VECTURA DELIVERY DEVICES LIMITED, Wiltshire (GB)

(21) Appl. No.: 18/561,536

(22) PCT Filed: May 16, 2022
(86) PCT No.: PCT/EP2022/063134

§ 371 Date: Nov. 16, 2023

(30) Foreign Application Priority Data
May 17, 2021 (EP) 21174149.1

(51) Int. Cl. A61M 15/00

(52) U.S. Cl. CPC A61M 15/008; A61M 15/0001; A61M 15/0045; A61M 2205/3306; A61M 2205/3327; A61M 2205/3331; A61M 2209/082; A61M 2230/40

(57) ABSTRACT

The invention provides an inhaler with a monitor having one or more sensors, wherein the monitor and the inhaler comprise a mounting mechanism that is configurable so that the monitor can be either detachably or permanently attached to the inhaler. The mounting mechanism may comprise a configurable male mounting member having a primary clip member and a secondary clip member, and a corresponding female mounting member which is a slot. The design provides both the removable and the fixed options, and only a simple additional step is necessary during assembly to select the desired configuration.

### TECHNICAL FIELD OF THE INVENTION

The present invention relates to an inhaler containing an active substance for inhalation and a monitor having one or more sensors for monitoring a patient's adherence and/or compliance. In particular, the invention relates to an inhaler with a monitor with a mounting mechanism that is configurable so that the monitor can be either detachably or permanently attached to the inhaler.

### BACKGROUND TO THE INVENTION

Inhalers, such as dry powder inhalers (DPIs), provide an attractive method for administering medicaments, for example to treat local diseases of the airway or to deliver drugs to the bloodstream via the lungs. The medicament is commonly provided as individual doses, such as a strip having a plurality of blisters. The dose is typically dispensed by the user opening a cap or cover to access a mouthpiece, then operating an actuator, such as a button or lever to release the powder, and finally inhaling through the mouthpiece. In some inhalers (known as “open-inhale-close” inhalers) the cover itself is the actuator, so that there is no separate actuating lever or button. The inhalers usually have a dose counter which displays the number of doses that have been used, or that remain to be used.

The efficacy of treatment is dependent on the patient using the inhaler correctly and as prescribed. Consequently, there is increasing interest in monitoring patient adherence (i.e. whether the patient takes the prescribed number of doses per day, e.g. once or twice daily) and compliance (i.e. whether the patient uses their inhaler correctly, e.g. if they inhale sufficiently strongly to entrain the powder and disperse it into particles that reach the lung).

DPIs typically contain a month's supply of medication. Since adherence/compliance monitors usually contain expensive sensors, electronics etc., they are often provided as separate add-on modules which detachably couple to the inhaler. Thus, when the medication in the inhaler has been used up, the monitor can be detached and then re-attached to a new inhaler. For example, WO 2014/204511 discloses adherence monitors for the Diskus® dry powder inhaler, which may be configured as a casing that fits over the top and bottom portions of the inhaler. The monitor is therefore easy to install and remove. WO 2015/178907 discloses a monitor in the form of an external casing into which the inhaler is inserted. The monitor has friction screws which are tightened in order to secure the monitor to the inhaler. The monitor is therefore removable by loosening the screws.

Alternatively, the monitor may be permanently attached to the inhaler, so that it cannot be inadvertently removed by the user. This ensures that the monitor records adherence/compliance information throughout the lifetime of the inhaler. For example, EP 3552647 discloses a built-in monitor for a Diskus®-type inhaler. The monitor is fixedly mounted to the casing of the inhaler under a cover unit by means of screws. EP 3551263 discloses an integrated monitor for a Diskus®-type inhaler which is located inside the housing of the inhaler. These monitors are not removable.

Thus both removable and permanently attached monitors are required, for different users and/or different products. It would be advantageous to provide a monitor for an inhaler which can be easily configured to be either permanently attached or removable without having to change the design of the monitor.

### BRIEF DESCRIPTION OF THE INVENTION

The present invention addresses this problem and, in a first aspect, provides an inhaler with a monitor having one or more sensors, wherein the monitor is attachable to the inhaler by a mounting mechanism comprising at least one pair of complementary mounting members, wherein one mounting member of the or each pair of mounting members is located on the inhaler and the other mounting member of the or each pair of mounting members is located on the monitor; wherein the mounting mechanism, in particular one or more of the mounting members, is configurable so that the monitor can be detachably mounted on the inhaler in a first configuration of the configurable mounting member(s) and can be permanently attached to (i.e. mounted onto) the inhaler in a second configuration of the configurable mounting member(s).

Each pair of mounting members may comprise a male mounting member and a female mounting member.

The monitor may comprise a male mounting member and the inhaler may comprise a female mounting member, or the inhaler may comprise a male mounting member and the monitor may comprise a female mounting member.

In contrast to the known monitors which are retro-fitted (either detachably or permanently) onto pre-existing inhalers, the inhaler and monitor of the present invention are designed from the start to be used with each other. This allows the mounting mechanism to be configurable so that the monitor can be attached to the inhaler in either a detachable or permanent manner. The single base design provides both the removable and the fixed options, and only a simple additional step is necessary during assembly to select the desired configuration.

In one embodiment, the monitor has two male mounting members and the inhaler has two female mounting members. One or both of the male mounting members may be configurable so that the monitor can be either detachably or permanently attached to (i.e. mounted onto) the inhaler.

The configurable male mounting member may be a clip comprising a primary clip member and a secondary clip member. The corresponding female mounting member may be a slot.

In the first configuration, when the monitor is detachably mounted on the inhaler, the primary clip member engages with the slot and the secondary clip member does not engage with the primary clip member and/or the slot. In the second configuration, when the monitor is permanently mounted on the inhaler, the primary clip member engages with the slot and the secondary clip member engages with the primary clip member and/or the slot.

The secondary clip member may be configurable, e.g. movable from a first position in which it does not engage with the primary clip member and/or the slot to a second position in which it engages with the primary clip member and/or the slot. The secondary clip member may be movable via an orifice in the housing of the inhaler or in the casing of the monitor, for example during assembly of the inhaler. Alternatively, the secondary clip member may be configurable by removing a breakable/detachable part.

The primary clip member may have a body and an enlarged head which acts as a pawl, and the corresponding slot may have a lip and a void, which forms a detent mechanism for holding the head of the primary clip member inside the slot so that the monitor can be mounted on the inhaler and can be detached from the inhaler with a small force (such as less than 30 N).

The secondary clip member may have a head and the primary clip member may have a step. When the monitor is permanently mounted on the inhaler, the head of the secondary clip member may engage with the step the primary clip member as a ratchet and pawl. The head of the secondary clip member may act as the pawl which engages with the step on the primary clip member that acts as the ratchet.

When the monitor is permanently mounted on the inhaler, the secondary clip member may prevent the primary clip member from moving so that the detent mechanism cannot be released. In particular, the head of the secondary clip member may be located inside the slot and may prevent the head of the primary clip member from overcoming the detent mechanism. Thus the monitor can only be detached from the inhaler with a large force (such as greater than 150 N).

The secondary clip member may have a flexible body. The secondary clip member may be a separate component. The secondary clip member may have a removable part.

In a second aspect, the invention provides a monitor for an inhaler, the monitor having one or more sensors, wherein the monitor has at least one configurable mounting member for attaching the monitor to the inhaler so that the monitor can be detachably mounted on the inhaler in a first configuration of the configurable mounting member(s) and can be permanently mounted on the inhaler in a second configuration of the configurable mounting member(s), wherein the at least one configurable mounting member comprises a primary clip member and a secondary clip member.

In a third aspect, the invention provides an inhaler for use with a monitor having one or more sensors, wherein the inhaler has at least one configurable mounting member for attaching the monitor to the inhaler so that the monitor can be detachably mounted on the inhaler in a first configuration of the configurable mounting member(s) and can be permanently mounted on the inhaler in a second configuration of the configurable mounting member(s), wherein the at least one configurable mounting member comprises a primary clip member and a secondary clip member.

### BRIEF DESCRIPTION OF THE FIGURES

The invention will now be further described with reference to the Figures, wherein:

FIG. 1A shows an inhaler and a monitor according to the invention, with the cover in the closed position, so that the mouthpiece is covered.

FIG. 1B shows the inhaler of FIG. 1A with the cover in the open position.

FIG. 1C shows the inhaler of FIG. 1A without the monitor and with the cover closed.

FIG. 1D shows the inhaler of FIG. 1A without the monitor and with the cover open.

FIGS. 2A and 2B show the monitor.

FIG. 3 shows a first clip.

FIGS. 4A, 4B and 4D show a second clip; FIG. 4C shows the orifice for configuring the second clip.

FIGS. 5A and 5B show a second embodiment of the second clip.

FIGS. 6A and 6B show a third embodiment of the second clip.

FIGS. 7A and 7B show a fourth embodiment of the second clip.

FIGS. 8A and 8B show a fifth embodiment of the second clip.

### DETAILED DESCRIPTION OF THE INVENTION

In the context of inhalers, the term “adherence” is normally used to refer to whether the patient takes the prescribed number of doses per day, e.g. once or twice daily. The term “compliance” is normally used to refer to whether the patient uses their inhaler correctly, e.g. if they inhale sufficiently strongly to entrain the powder and disperse it into particles that reach the lung. Consequently, a monitor may be designed to measure adherence and/or compliance, according to the type of sensors that it uses, and how they are configured. In the present application, the term “monitor” therefore refers to a module having one or more sensors that is designed to measure and capture information relating to adherence and/or compliance. However, the monitor does not perform any of the functions associated with dosing the medication, such as a piercing or opening blisters/capsules, de-agglomerating the powder or providing a breath-actuation mechanism. The inhaler therefore operates to dispense the active substance whether the monitor is present or not.

The term “mounting mechanism” refers to a mechanism for mechanically connecting the inhaler and monitor using at least one pair of mounting members with complementary shape such as to engage with each other, wherein one mounting member of each pair is located on the inhaler, and the other is located on the monitor. One of the members (often referred to as “male”) is inserted, clicked or hooked into, and/or caught by, the other member (often referred to as “female”). A male member has a particular shape (such as as a peg or other protrusion) adapted for mechanical engagement with a complementary female member (such as a slot or other cavity). Thus the mounting mechanism locates the monitor in a pre-defined position with respect to the inhaler and provides a retaining force to hold it in position. For example, the mounting mechanism may comprise a clip (male feature) in the form of protrusion with an enlarged head and a corresponding slot (female feature) with a lip. Typically, the male member is somewhat flexible, and, in the detachable configuration, release is easy and achieved e.g. by slightly pressing or pulling the monitor, such as in the case of a snap-fit connection. In the permanent configuration, the movement of the male member is restricted so that it cannot flex, thereby preventing detachment of the monitor.

The term “detachably attached” means that the monitor is intended to be removable by a user, for example using their fingers. This term does not refer to monitors that are intended not to be removable by a user, and which are only removable by applying a very large force, such as with a tool. The terms “removable” and “detachable” are used interchangeably.

The term “permanently attached” means that the monitor is not intended to be removable by a user, for example using their fingers. However, this term does not require that it is completely impossible to remove the monitor (so for example, a permanently attached monitor might be removable by using tools).

The term “configurable” refers to a mounting member, or component thereof, which is designed to have two different positions or states. The state can be selected or changed, in particular during assembly of the inhaler and the monitor. The selection or change can be effected for example by moving (such as bending, flexing, translating) the component; or by removing (such as breaking off) a detachable part of the component.

The inhaler of the invention preferably has a strip of blisters containing powdered medicament, and a mechanism for advancing the blister strip and for opening the blisters which is operated by an actuator. The opening mechanism is suitably a piercer which is mounted on the underside of the mouthpiece. The actuator drives the indexing mechanism to move a blister into alignment with the piercer and then moves the mouthpiece relative to the housing so that the piercer pierces the aligned blister. The actuator may be a lever which causes indexing of the blister strip and piercing of the blisters. Preferably however, the actuator is formed as part of, or is connected to, the cover, so that rotation of the cover causes indexing of the blister strip and piercing of the blisters. The inhaler may be configured to index and pierce one blister on each actuation. Alternatively, it may index and pierce two (or more) blisters on each actuation. For example, it may deliver two (or more) different formulations or medicaments simultaneously.

However, the invention is not limited to this type of inhaler, and for example, could equally be an inhaler which has a passive mouthpiece cover, and a separate actuating lever, as described for example in WO13/175176, or with an inhaler which has a blister disk instead of a blister strip, or a reservoir DPI or a capsule DPI. Moreover, the invention can equally apply to other types of inhaler, such as pressurized metered dose inhalers (pMDI) or soft mist inhalers.

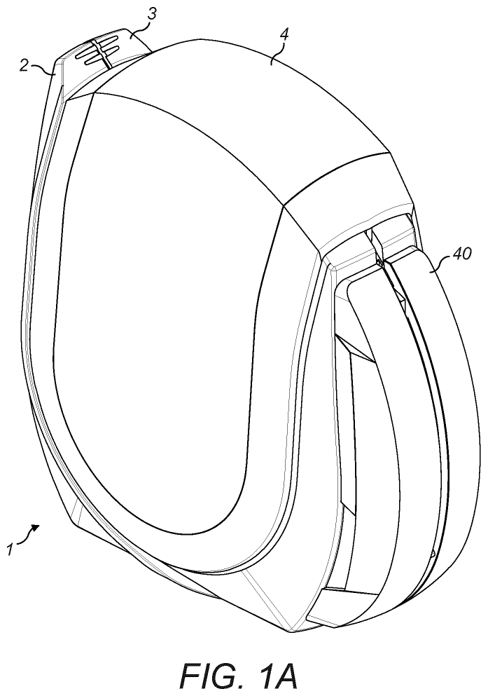

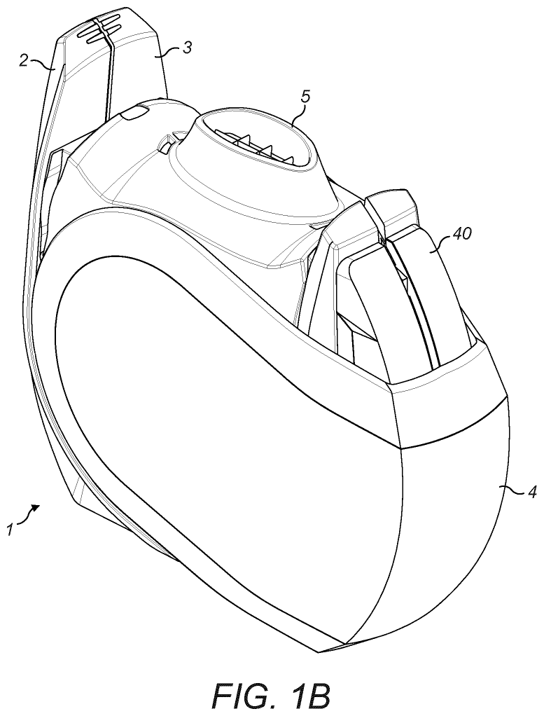

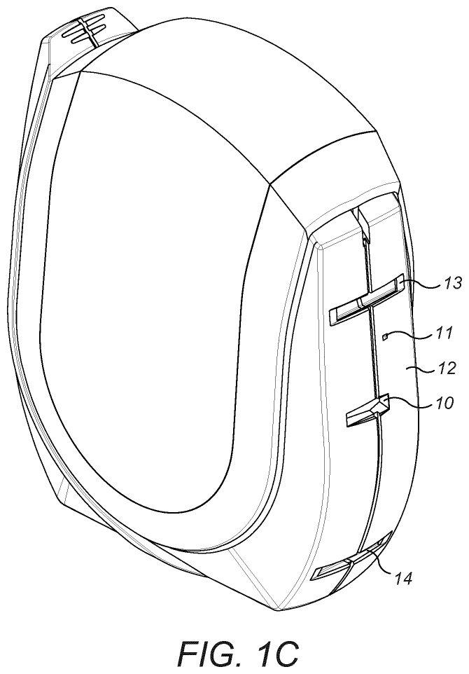

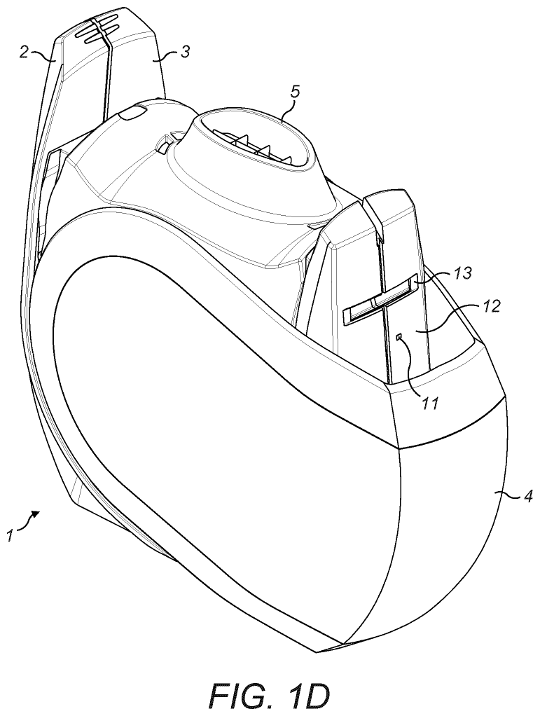

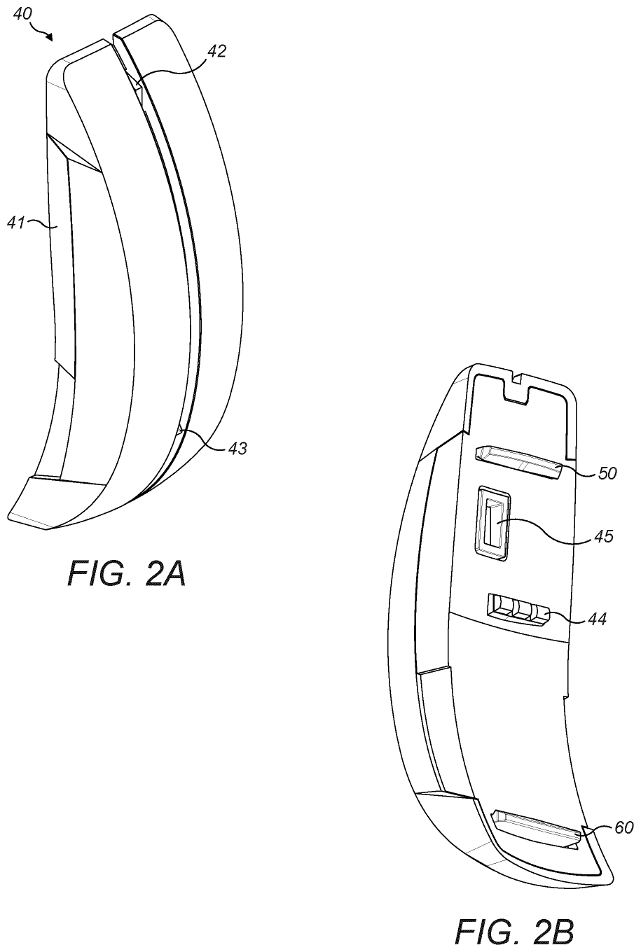

An inhaler and monitor of the invention are shown in FIGS. 1 and 2. FIG. 1A shows the inhaler with a monitor attached, and with the mouthpiece cover in the closed position. FIG. 1B shows the inhaler with the mouthpiece cover in the open position so that the mouthpiece is visible. FIGS. 1C and 1D show the inhaler with the monitor removed and with the mouthpiece cover in the closed and open positions respectively.

The inhaler shown in FIG. 1 is an “open-inhale-close” dry powder inhaler of the type described in WO13/175177, which has a gear mechanism that selectively couples the mouthpiece cover to a blister strip indexing mechanism and also to a piercer. Moving the cover from the closed position to an intermediate position causes the indexing mechanism to advance the blister strip. Then, once an unused blister is in position beneath the piercer, the indexing mechanism is disengaged. Moving the mouthpiece cover from the intermediate position to the fully open position causes the piercer to pierce the aligned blister. The user then inhales through the mouthpiece, which aerosolizes the powder in the pierced blister.

The inhaler 1 shown in FIG. 1 is constructed from two shell parts 2, 3 which are joined together to form a housing that contains a blister strip. A monitor 40 is attached to one side of the inhaler. A mouthpiece cover 4 is mounted onto the housing. The cover 4 can be rotated through approximately 100° from the closed position (FIG. 1A) in which it covers and protects a mouthpiece, to a fully open position (FIG. 1B), in which the mouthpiece 5 is exposed so that the user can inhale a dose of medicament. When the cover is in the open position for inhalation, the monitor is situated under the cover, so that it is protected from accidental damage.

The inner face of the monitor matches the shape of the housing of the inhaler on which it is mounted. The outer face corresponds to the curve defined by the rotation of cover. In other words, it is shaped as an arc of a circle centred on the axis of rotation of the cover, with a radius which is slightly less than the radius of the internal surface of the cover. Thus, when the cover is opened there is a small clearance gap (about 0.5-1 mm) between the internal surface of the cover and outer face of the monitor. This maximises the volume of the monitor within the constraint that it can fit under the cover.

FIGS. 1C and 1D show the inhaler without the monitor. The inhaler has two slots 13, 14 for mounting the monitor (the second slot 14 is not visible in FIG. 1D because it is hidden by the cover 4). There are two orifices 10, 11 in the wall 12 of the housing where the monitor was attached whose purposes are described below.

FIGS. 2A and 2B show the monitor 40, which has a casing 41 containing a circuit board and one or more sensors. The outside face of the monitor shown in FIG. 2A has a sensor or switch 42 which is used to determine whether the mouthpiece cover has been opened. It also has an orifice 43 whose purpose is explained below.

The inner face of the monitor (i.e. the side which abuts the inhaler when the monitor is attached) is shown in FIG. 2B. The monitor has three optical sensors 44 which protrude into the orifice 10 on the inhaler when the monitor is attached to the inhaler. The optical sensors read a code on the blister strip (for example, a printed bar code) so that the monitor can determine the number of doses that have been dispensed or that remain to be dispensed. The monitor also has a pressure sensor 45, which is located in a recess on the inner face. The pressure sensor abuts the orifice 11, which leads, via a channel in the housing, to the mouthpiece. The monitor can measure the pressure in the mouthpiece and thereby detect the user's inhalation. The monitor 40 also has two clips 50, 60 which fit into the corresponding slots 13, 14 in the housing of the inhaler, and thereby hold the monitor in place when attached to the inhaler. The first (upper) clip 50 is stiff and the second (lower) clip 60 is flexible (although equally the upper clip could be flexible and the lower clip could be stiff).

The monitor may be configured so that it is detachable and hence may be transferred to a new inhaler once the medication in the original inhaler has been used up. In this detachable configuration, the monitor can be removed with a force of less than 30 N, which is low enough for elderly or infirm users to be able to detach it. Alternatively, the monitor may be configured so that it is permanently attached to the inhaler. In this fixed configuration, the monitor is capable of resisting attempted removal by a user, and requires a force of e.g. greater than 150 N to remove it. These two configurations are achieved from a single design by designing the second clip 60 so that it can be configured during the factory assembly process in either the permanently mounted state or the detachably mounted state.

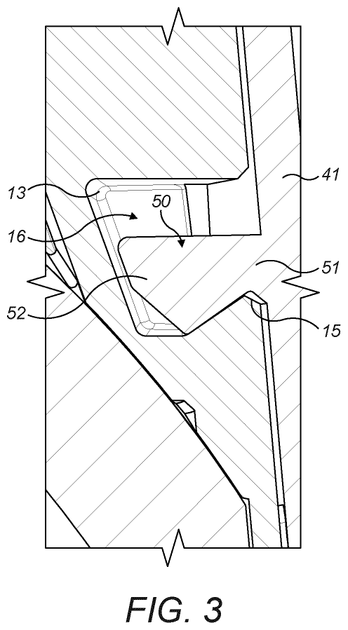

FIG. 3 is a cross-sectional view through the inhaler and the monitor in the region of the first slot 13 and the first clip 50. It shows the first clip 50 in place in the first slot 13 when the monitor is attached to the inhaler. The first clip 50 protrudes from the casing 41 of the monitor, and has a neck 51 and an enlarged head 52. The first slot 13 has a lip 15 on its lower side and a void 16. To attach the monitor to the inhaler, the head 52 is first inserted into the slot 13 over the lip 15. Then it is pulled downwards in order to line up the second clip with the second slot for insertion (described below) so that the head 52 is located behind the lip 15. The first clip 50 is rigid, so that the lip 15 retains the head 52 in place. If the second clip is released from the second slot, the head 52 is then free to rise up over the lip 15 and the first clip 50 can be removed from the first slot 13.

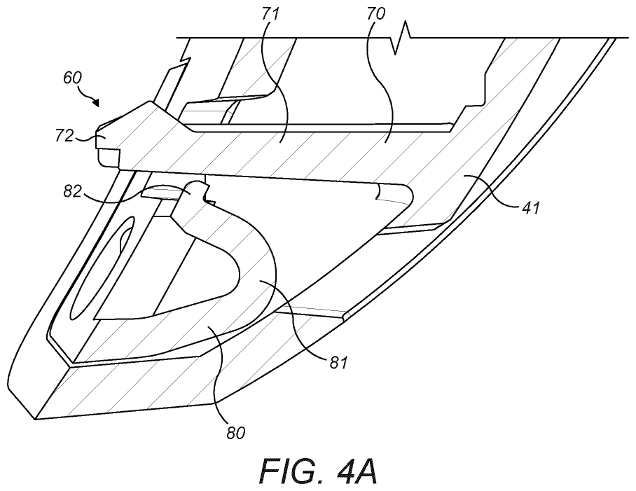

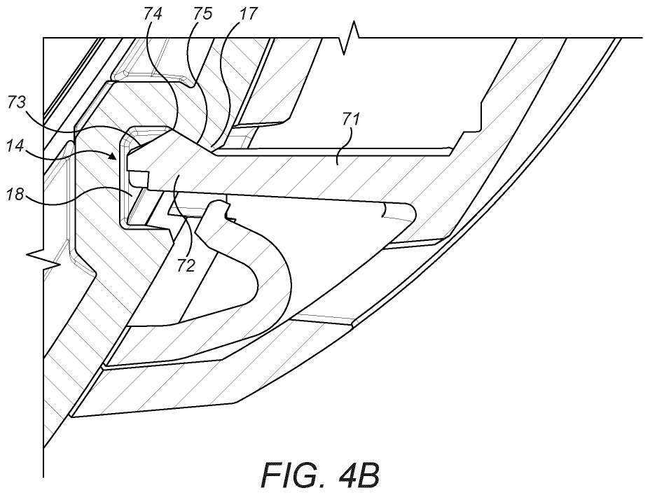

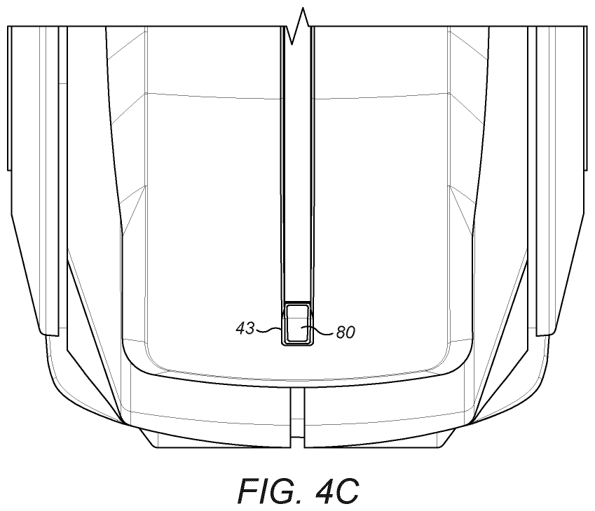

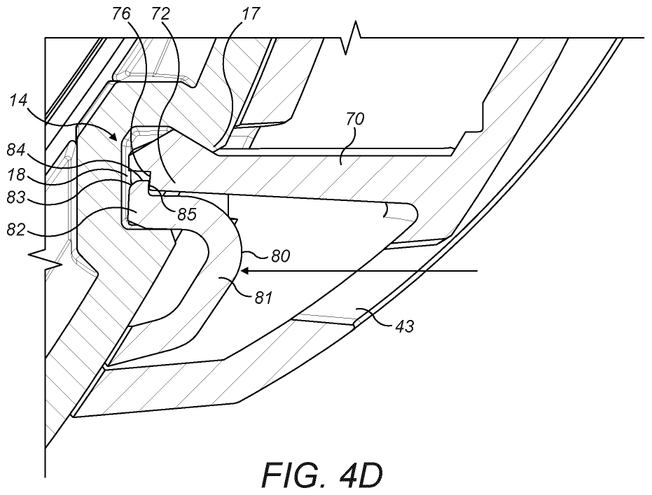

FIG. 4A is a cross-sectional view through the lower part of the monitor showing the second clip 60 in detail. The second clip 60 has a primary clip member 70 with a generally planar body 71 and an enlarged head 72. The body 71 is located inside the casing 41 of the monitor, and the head 72 protrudes outside the casing. The second clip 60 also has a secondary clip member 80 with an arcuate body 81 and a head 82, also located inside the casing. The secondary clip member 80 is flexible due to the arcuate shape of the body so that the head 82 can be moved into a position in which it protrudes outside of the casing, in a manner that is explained below.

FIG. 4B shows the second clip engaged with the second slot in the removable configuration. In this configuration, the secondary clip member is passive and remains inside the casing of the monitor. The second slot 14 has a lip 17 on its upper side and a void 18. When the monitor is attached to the inhaler, the head 72 of the primary clip member 70 is inserted into the slot 14. The head 72 has an inverted “V” shaped profile, so that the front face 73 of the head slopes upwards to a ridge 74, and the rear face 75 of the head slopes downwards from the ridge 74. The body 71 is resilient, so that as the head 72 is inserted into the slot 14, the lip 17 pushes against the sloping front face 73, and the head 72 is deflected downwards into the void 18, until the lip 17 passes over the ridge 74. The resilience of the body 71 causes the head to deflect back upwards as the rear face 75 moves over the lip. Thus the lip 17 together with the resilient body 71 provides a detent mechanism for holding the head 72, which acts as a pawl, inside the slot 14. This, together with the first clip 50 in the first slot 13, holds the monitor in place on the inhaler during use.

In this configuration, the insertion process is reversible: as the head 72 is removed from the slot 14, the lip 17 pushes against the sloping rear face 75, the head 72 is deflected downwards into the void 18, until the ridge 74 rides over the lip 17. The resilience of the body 71 causes the head to deflect back upwards as the front face 73 moves over the lip. Thus sufficient force must be applied to the monitor for the body 71 to be deflected so that the head 72 passes over the lip, i.e. in order to overcome the hold placed on the head by the detent mechanism. Once the second clip has been released from the second slot, the monitor can be moved upwards a short distance so that the first clip can be removed from the first slot. In this manner, the monitor can be detached from the inhaler with a relatively small force (such as less than 30 N), e.g. once all of the doses have been inhaled.

FIG. 4C shows the lower part of the outer side of the monitor 40 attached to the inhaler. The secondary clip member 80 is visible through the orifice 43. In order to change from the detachably mounted configuration to the permanently attached configuration, a rod is inserted though the orifice 43 (e.g. during assembly of the inhaler) so that it comes into contact with and pushes against the body 81 of the secondary clip member. The secondary clip member flexes so that the head 82 is moved out of the casing 41 of the monitor and into the second slot 14.

FIG. 4D shows the second clip engaged with the second slot in the fixed (i.e. permanently attached) configuration. The head 72 of the primary clip member 70 is inserted behind the lip 17 of the slot 14 as described above for the detachable configuration. Then, the secondary clip member 80 is pushed via the orifice 43 (indicated by the arrow) so that the head 82 is inserted into the void 18 beneath the head 72 of the primary clip member 70. The lower side of the head 72 of the primary clip member 70 has a step 76. The front face 83 of the head 82 of the secondary clip member 80 slopes upwards to a plateau 84, and the rear face 85 forms a step downwards from the plateau 84. The arcuate body 81 is flexible, so that as the head 82 is inserted into the void 18, the sloping front face 83 pushes against the lower side of the head 72 of the primary clip member 70. This deflects the head 82 downwards, until it reaches the step 76. Once the head 82 has passed over the step 76, the resilience of the arcuate body 81 causes the head 82 to deflect upwards into the void 18 beyond the step 76. The head 82 is then located behind the step 76. Since the rear face 85 of the head is vertical (unlike the rear face 75 of the head 72 of the primary clip member 70), the head 82 is held behind the step 76. Thus the step 76 on the head 72 of the primary clip member 70 and the head 82 of the secondary clip member 80 provide a ratchet and pawl. The body 81 of the secondary clip member 80 is located beneath the head 72 of the primary clip member 70 and prevents the head 72 from being deflected downwards if the user attempts to remove the monitor from the inhaler. In other words, the secondary clip member 80 is locked in place by the head 82 and the step 76; this in turns holds the head 72 of the primary clip member 70 in place behind the lip 17. A much larger force (such as greater than 150 N) is thus required to remove the monitor, so that it is essentially permanently attached.

In an alternative arrangement, the step 76 could be provided on the lower surface of the slot 14, and the head 82 of the secondary clip member 80 could protrude downwardly so that when the secondary clip member 80 is inserted into the slot 14, the head 82 would again be held behind the step 76.

In summary, when the secondary clip member is not inserted into the slot (FIG. 4B), the second clip is able to flex, which permits removal of the monitor with a force of less than 30 N. To change to the fixed configuration, the secondary clip member is inserted into the slot, for example during the assembly process after the monitor has been attached. This locks the second clip in place so that it is no longer able to flex, thereby preventing removal of the monitor. Thus the second clip can be easily configured so that the force required to remove the monitor is greater than 150 N.

The user should not be able to inadvertently or misguidedly change from the detachable configuration to the fixed configuration whilst using the monitor. Thus the secondary clip member should be sufficiently flexible that it can be pushed into the fixed configuration during assembly, but sufficiently stiff that a user is not able to push it into the fixed configuration by for example, poking a pin into the aperture. Suitably, the force required to push the secondary clip member into the fixed configuration is greater than 300 N.

The monitor can be attached to the inhaler by inserting the first clip into the first slot, and then the second clip into the second slot as described above. Alternatively, the second clip can first be inserted into the second slot, and then subsequently the first clip can be inserted into the first slot. If the monitor is to be permanently attached to the inhaler, the second clip is placed into the fixed configuration by pushing the secondary clip member into position in the second slot via the orifice.

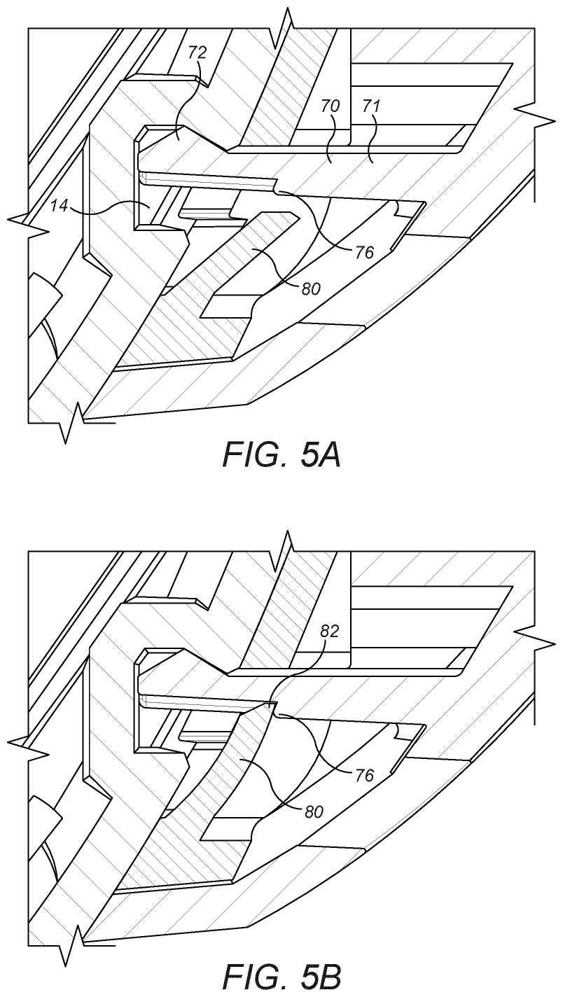

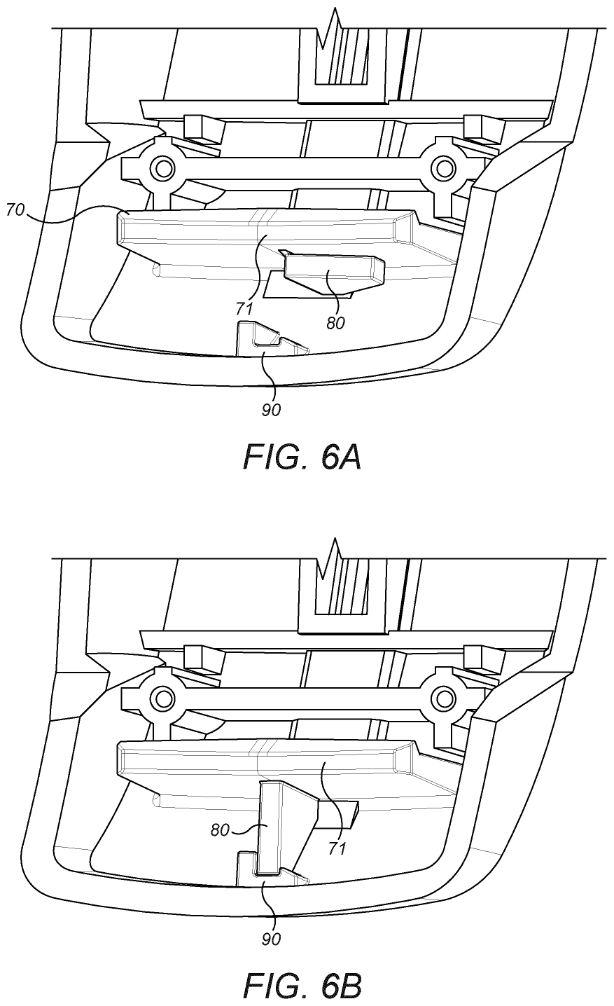

The secondary clip member could interact with the primary clip member outside the slot rather than inside it, as shown in FIGS. 5 and 6. In the embodiment of FIG. 5, the primary clip member 70 has a step 76 located on its body 71 rather than on the head 72, and the secondary clip member 80 is movable from a position in which it does not interact with the primary clip member 70 (the detachable configuration, shown in FIG. 5A) into a position in which its head 82 engages with the step 76 and holds the primary clip member 70 in place (the permanently attached configuration, as shown in FIG. 5B) without entering the slot 14. FIG. 6 shows an alternative embodiment in which the secondary clip member 80 is a living hinge which is rotated from a position in which it does not interact with the primary clip member 70 (the detachable configuration, shown in FIG. 6A) into a position in which it holds the body 71 of the primary clip member 70 in place (the permanently attached configuration, shown in FIG. 6B). Instead of a step on the primary clip member, the casing of the monitor has a detent feature 90 which holds the secondary clip member 80 in place.

In an alternative embodiment, the secondary clip member is a separate component rather than an integral part of the second clip. Thus, when assembling the monitor for the detachable configuration, the secondary clip member is not present at all. On the other hand, when assembling the monitor for the fixed configuration, the secondary clip member is inserted into the slot. The secondary clip member may be included in the clip assembly before mounting the monitor onto the inhaler and then inserted into the slot, for example by pushing a rod through the aperture to move the secondary clip member into position in the slot. Alternatively, the secondary clip member could itself be inserted through the aperture and into the slot. Since the secondary clip member is not present at all in the detachable configuration, there is no possibility of the user inadvertently changing from the detachable configuration to the fixed configuration whilst using the monitor, even if the user poked a pin into the aperture.

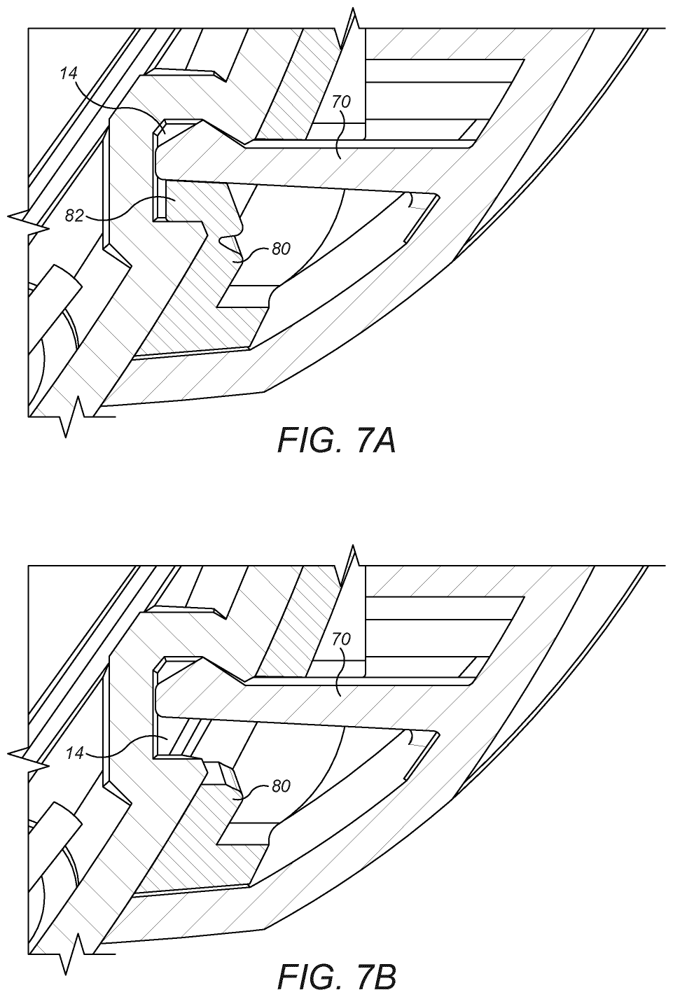

Instead of the secondary clip member being movable, it could be rigid (so that it is not movable), but have a part which can be broken off or otherwise removed before or during assembly in order to change from the permanently attached configuration to the removable configuration. For example, in the embodiment shown in FIG. 7, the secondary clip member 80 has a detachable head 82. When the is present (FIG. 7A), it locks the primary clip member 70 in place in the slot 14 so that the monitor is permanently mounted on the inhaler. When the head has been removed (FIG. 7B), there is nothing to lock the primary clip member 70 in the slot 14, so that the monitor is detachably mounted on the inhaler.

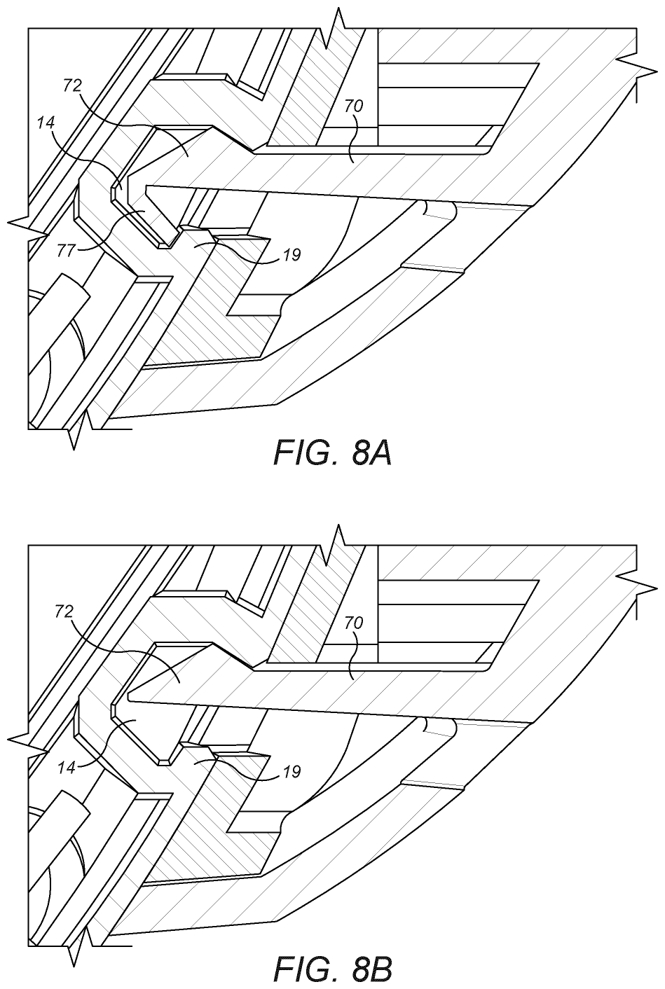

In yet another embodiment, shown in FIG. 8, there is no secondary clip member; instead, the head 72 of the primary clip member 70 has a removable tooth 77 which, when present, engages with a lip 19 on the lower side of the slot 14 to lock the primary clip member 70 in place (FIG. 8A), so that the monitor is permanently attached to the inhaler. Similar to the previous embodiment, removing the tooth before assembly prevents the primary clip member 70 from being locked in the slot 14 (FIG. 8B), so that the monitor is detachably mounted.

In the embodiment shown in FIGS. 1 to 4, there are two pairs of mounting members. However, the inhaler and monitor could have only one pair of mounting members (e.g. one male mounting member, such as a clip, and one female mounting member, such as a slot); alternatively, the inhaler and monitor could have three or more pairs of mounting members.

In the embodiment shown in FIGS. 1 to 4, the monitor has the clips (male mounting members) and the inhaler has the slots (female mounting members). This has the advantage that there are no clips protruding from the inhaler, so that the inhaler is more comfortable to hold if it is used without the monitor. Moreover, it is easy to access the secondary clip member through the aperture in the casing of the monitor in order to place the second clip into the fixed configuration. Nonetheless, the monitor could instead have the slots and the inhaler could have the clips.

It is only necessary for one of the mounting members to be configurable; nonetheless several or all of the mounting members may be configurable. For example, in the embodiment described above, only the second (lower) clip is configurable. However, the first (upper) clip could alternatively or additionally be configurable. Having more than one configurable mounting member has the advantage that an even greater force would be required to remove the monitor in the fixed configuration; however, increasing the number of configurable mounting members may increase the cost and the complexity of assembly.

In the embodiment shown in FIGS. 1 to 4, the first (non-configurable) clip and the first slot help to retain the monitor in place by means of the enlarged head of the clip and the lip of the slot. This is advantageous, since having two pairs of mounting members which retain the monitor in place provides for secure attachment (in either the detachable or the fixed configuration). However, this is not essential and in another embodiment, the first clip and slot could simply help to position the monitor in place on the inhaler without providing any retention. For example, the clip could be a simple protrusion (without an enlarged head) and the slot could have no lip. In this embodiment, only the second (configurable) clip would hold the monitor on the inhaler.

In the embodiment shown in FIGS. 1 to 4, the configurable mounting member is a male mounting member. However, a female mounting member could alternatively or additionally be configurable, for example by having a flexible or movable secondary slot member which could be moved into the void in order to lock the primary clip member in place.

The invention provides a monitor which can be either detachably or permanently attached to an inhaler using a simple assembly process. Creating the permanent configuration needs only a simple additional step (e.g. pushing a component with a rod) and does not require, for example, a welding or gluing step.

The monitor may have a controller and memory (e.g. a suitable microprocessor) which are configured to process and/or store information read from the sensors relating to patient's usage of the inhaler. The monitor may also include communication means to transmit adherence/compliance information (e.g. via bluetooth) to an external device, such as a computer or smartphone. The information may then be displayed to the patient and/or a medical professional, by means of suitable software, for example a smartphone app. The information may additionally or alternatively be stored on the monitor for subsequent interrogation, or wirelessly transmitted to, for example, an online health platform.

The medicament is suitable for administration by inhalation, for example for the treatment of a respiratory disease. It may include one of more of the following classes of pharmaceutically active material: anticholinergics, adenosine A2A receptor agonists, 2-agonists, calcium blockers, IL-13 inhibitors, phosphodiesterase-4-inhibitors, kinase inhibitors, steroids, CXCR2, proteins, peptides, immunoglobulins such as Anti-IG-E, nucleic acids in particular DNA and RNA, monoclonal antibodies, small molecule inhibitors and leukotriene B4 antagonists. The medicament may include excipients, such as fine excipients and/or carrier particles (for example lactose), and/or additives (such as magnesium stearate, phospholipid or leucine).

Suitable B2-agonists include albuterol (salbutamol), e.g. albuterol sulfate; carmoterol, e.g. carmoterol hydrochloride; fenoterol; formoterol; milveterol, e.g. milveterol hydrochloride; metaproterenol, e.g. metaproterenol sulfate; olodaterol; procaterol; salmeterol, e.g. salmeterol xinafoate; terbutaline, e.g. terbutaline sulphate; vilanterol, e.g. vilanterol trifenatate or indacaterol, e.g. indacaterol maleate. Suitable steroids include budesonide; beclamethasone, e.g. beclomethasone dipropionate; ciclesonide; fluticasone, e.g. fluticasone furoate; mometasone, e.g. mometasone furoate. Suitable anticholinergics include: aclidinium, e.g. aclidinium bromide; glycopyrronium, e.g. glycopyrronium bromide; ipratropium, e.g. ipratropium bromide; oxitropium, e.g. oxitropium bromide; tiotropium, e.g. tiotropium bromide; umeclidinium, e.g. umeclidinium bromide; Darotropium bromide; or tarafenacin.

The active material may include double or triple combinations such as salmeterol xinafoate and fluticasone propionate; budesonide and formoterol fumarate dehydrate; glycopyrrolate and indacaterol maleate; glycopyrrolate, indacaterol maleate and mometasone furoate; fluticasone furoate and vilanterol; vilanterol and umeclidinium bromide; fluticasone furoate, vilanterol and umeclidinium bromide.

1. An inhaler having a housing and a monitor having a casing and one or more sensors, wherein the monitor is attachable to the inhaler by a mounting mechanism comprising at least one pair of complementary mounting members, wherein the inhaler comprises one mounting member of the or each pair of mounting members and monitor comprises the other mounting member of the or each pair, wherein at least one of the mounting members is configurable so that the monitor can be detachably mounted on the inhaler in a first configuration of the configurable mounting member(s) and can be permanently mounted on the inhaler in a second configuration of the configurable mounting member(s).

2. The inhaler with a monitor according to claim 1 wherein the or each pair of mounting members comprise a male mounting member and a female mounting member.

3. The inhaler with a monitor according to claim 1, wherein the monitor has two male mounting members and the inhaler has two female mounting members, and wherein one of the male mounting members is configurable.

4. The inhaler with a monitor according to claim 3, wherein the configurable male mounting member comprises a primary clip member and a secondary clip member, and the corresponding female mounting member is a slot.

5. The inhaler with a monitor according to claim 4, wherein:
in the first configuration the primary clip member engages with the slot and the secondary clip member does not engage with the primary clip member and/or the slot, so that the monitor is detachably mounted on the inhaler; and wherein
in the second configuration the primary clip member engages with the slot and the secondary clip member engages with the primary clip member and/or the slot, so that the monitor is permanently mounted on the inhaler.

6. The inhaler with a monitor according to claim 4, wherein the secondary clip member is movable from a first position in which it does not engage with the primary clip member and/or the slot to a second position in which it engages with the primary clip member and/or the slot.

7. The inhaler with a monitor according to claim 6 wherein the secondary clip member is movable via an orifice in the housing of the inhaler or in the casing of the monitor.

8. The inhaler with a monitor according to any of claim 4, wherein the primary clip member has a body and a head, and the corresponding slot has a lip and a void, which together form a detent mechanism for holding the head of the primary clip member inside the slot.

9. The inhaler with a monitor according to claim 8, wherein, when the monitor is permanently mounted on the inhaler, the secondary clip member prevents the primary clip member from moving so that the detent mechanism cannot be released.

10. The inhaler with a monitor according to claim 9, wherein the secondary clip member has a head, and wherein when the monitor is permanently
